(19) United States
(12) Patent Application Publication
(10) Pub. No.: US 2006/0165618 A1
(43) Pub. Date: Jul. 27, 2006

(54) Photoactivatable composition and uses thereof

(75) Inventors: Gabin Vic, Compiegne (FR); Aude Livoreil, Paris (FR)

Correspondence Address: STEPTOE & JOHNSON LLP, 1330 CONNECTICUT AVENUE, N.W., WASHINGTON, DC, 20036, US

(73) Assignee: L'Oreal, Paris (FR)

(21) Appl. No.: 11/325,338

(22) Filed: Jan. 5, 2006

Related U.S. Application Data
(62) Division of application No. 10/279,757, filed on Oct. 25, 2002.

(30) Foreign Application Priority Data
Oct. 29, 2001 (FR) 0113970

(51) Int. Cl. A61K 8/73; A61K 8/49; A61K 8/40

(52) U.S. Cl. 424/59; 424/63; 424/70.13

(57) ABSTRACT

The invention relates to a cosmetic composition including a photoactivatable compound having a support molecule chosen from carbon-containing and/or sulfur-containing and/or nitrogen-containing and/or phosphorus-containing nonpolymer compounds, polymers and oligomers and to which are covalently bonded at least two species containing chemical groups which, after irradiation between 200 et 450 nm, are converted into reactive species capable of reacting with chemical groups belonging to other molecules.

This application is a divisional of application Ser. No. 10/279,757 filed Oct. 25, 2002.

The present invention relates to a photoactivatable compound comprising a support molecule chosen from carbon-containing and/or sulfur-containing and/or nitrogen-containing and/or phosphorus-containing nonpolymer compounds, and to polymers and oligomers to which are covalently bonded at least two photoactivatable functional groups.

The invention also relates to cosmetic compositions containing such photoactivatable compounds and to cosmetic treatment processes using such photoactivatable compounds.

The products generally used to fix cosmetic agents onto keratin materials have the drawback of being very rapidly eliminated by washing or during other treatments.

Thus, the inventors of the present patent application have investigated a composition allowing the long-lasting fixing of cosmetic agents to keratin materials.

The prior art discloses coloring processes using photoactivatable compounds as coloring agents, and thus the process for coloring keratin materials described in patent application FR 2 605 220 consists in placing the keratin materials in contact with an aromatic azide or an azidoindole and then exposing said keratin materials to an appropriate light source to develop the color.

The prior art also discloses processes for producing deposits of materials on hair fibers. These processes require the presence of reactive groups on the material to be deposited, that can react with reactive groups of the hair fibers. These are multi-step processes during which the material to be deposited, on the one hand, and the hair fibers, on the other hand are modified in order for the activated sites of both parts to be able to interact. U.S. Pat. No. 5,211,942 may be mentioned in this regard.

The aim of the present patent application is to propose compounds that allow, preferably in a single step, the long-lasting fixing of a cosmetic agent to keratin materials. These compounds provide bonding between the cosmetic agent and the keratin materials: they are fixed to the cosmetic agent and to the keratin materials by covalent grafting either simultaneously or sequentially.

One subject of the present invention is a photoactivatable compound comprising a support molecule, with or without cosmetic activity, chosen from carbon-containing and/or sulfur-containing and/or nitrogen-containing and/or phosphorus-containing nonpolymer compounds, and to polymers and oligomers and to which are covalently bonded at least two species (photoactivatable functional groups) containing chemical groups which, after irradiation between 200 and 450 nm, are converted into reactive species capable of reacting with chemical groups belonging to other molecules.

A subject of the invention is also a cosmetic composition comprising at least one photoactivatable compound according to the invention.

A subject of the invention is also a cosmetic composition comprising at least one photoactivatable compound according to the invention and at least one cosmetic active agent. In the latter case, the photoactivatable compound and the cosmetic active agent may be covalently bonded or may be present in the form of a mixture.

The cosmetic compositions according to the present invention comprise a cosmetically acceptable solvent such as water, ethanol or mixtures thereof. This solvent may also contain other organic solvents such as C₅ to C₁₀ alkanes, acetone, methyl ethyl ketone, methyl acetate, ethyl acetate, butyl acetate, dimethoxyethane or diethoxyethane, and mixtures thereof.

According to one variant of these two compositions, the photoactivatable compound comprises a support molecule with cosmetic activity.

The cosmetic composition may be used as an agent for coloring the skin, the nails or the hair, as a moisturizer, as an agent for increasing the sheen, especially of the hair (sheen agent), as a sunscreen, as a conditioner or as an agent for shaping keratin fibers.

Another subject of the invention comprises a cosmetic process for treating keratin materials, preferably the hair, which consists in applying to these keratin materials a cosmetic composition according to the invention in one step or in several successive steps, and then in exposing the keratin materials to irradiation of one or more wavelengths of between 200 and 450 nm, preferably 200 and 400 nm and more preferably between 250 and 400 nm.

The expression “keratin materials” means the hair, the eyelashes, the eyebrows, bodily hairs, the nails or the skin.

As support molecules that may be used to prepare the photoactivatable compound according to the invention, carbon-containing and/or sulfur-containing and/or nitrogen-containing and/or phosphorus-containing nonpolymer compounds, and polymers and oligomers may be used.

The expression “carbon-containing nonpolymer compound” means a linear or branched or cyclic chain, containing from 1 to 80 carbon atoms and preferably 1 to 50 carbon atoms, optionally comprising 1 to 25 and preferably 1 to 10 hetero atoms (O, N, Si, S or P), this chain optionally being substituted with 1 to 60 substituents chosen from hydroxyl, amine, thiol, carbamate, ether, acid, ester, amide, cyano, ureido or halogen (in particular fluorine or chlorine) groups.

Examples of sulfur-containing compounds that may be mentioned include sulfur oxides and disulfides.

The term “polymer” means any natural or synthetic polymer that may be used in cosmetics, and in particular the polymers obtained by free-radical polymerization or by polycondensation or by opening rings. These polymers may be linear, branched or starburst polymers.

Natural polymers that will preferably be used include polysaccharides (for example dextrans, celluloses, starches, chitosan, pullulan, insulin, carageenan, guar, alginates, xanthans and hyaluronic acid), and proteins such as albumin, ovalbumin, keratin and collagen.

The natural polymers may be chemically modified; at least one group chosen from hydroxyalkyl, carboxyalkyl, amino, thio and aldehyde or epoxy functions may thus be introduced into the main chain of this natural polymer.

The synthetic polymers may be homopolymers or copolymers.

Polyurethanes, polyureas, polyethers, polyesters or polyamides will preferably be used.

They may also be dendritic polymers or dendrimers, as described by D. A. Tomalia et al., Angewandlte Chemie, Int. Engl. Ed., vol. 29, no. 2, pp. 138-175. These dendrimers are molecular structures constructed around a generally multivalent central unit. Around this central unit are attached sequences of branched chain-extending units in concentric layers and in a fully defined structure, thus giving rise to monodispersed symmetrical macromolecules of well-defined chemical and stereochemical structure.

They may also be dendritic polymers such as hyperbranched polymers. Examples of these polymers are described in patent applications WO 93/17060 and WO 96/12754. The part of the patent applications relating to these hyperbranched polymers and to their synthesis is expressly incorporated herein by reference.

They may also be dendrons as defined in the article by D. A. Tomalia.

At least two of the substituents of the support molecule must be capable of reacting with photoactivatable functional groups to give the photoactivatable compound according to the present patent application.

Included among the photoactivatable functional groups are especially diazirines, phenyl azides or aryl azides, diazo compounds such as diazoacetates and diazopyruvates, benzophenones, benzyl thio ethers, and substituted nitrobenzenes that can generate reactive species (carbenes, nitrines, free-radical species, etc.) by simple exposure to radiation of one or more wavelengths of between 200 and 450 nm, preferably 200 and 400 nm and even more preferably 250 and 400 nm. This exposure may be performed via solar simulators, UV lamps or by the simple action of sunlight. These reactive species have the property of being able to unselectively insert themselves into many chemical bonds such as C—H, N—H, O—H, C—C, C═C, S—H or C≡C bonds of the keratin material or cosmetic agent.

The diazirines that may be used for the purpose of the present patent application are, for example, preferably molecules having the formula:

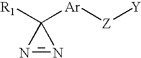

in which R₁ is chosen from the group formed by a hydrogen atom, a linear or branched C₁ to C₁₀ alkyl radical, a linear or branched C₂ to C₁₀ alkenyl radical, a linear or branched C₂ to C₁₀ alkynyl, CF₃, CCl₃, CBr₃, NR′₃+, SR′₂+, SH₂+, NH₃+, NO₂, SO₂R′, C≡N, COOH, F, Cl, Br, I, OR, COOR′, SO₃H, COR′, SH, SR′ and OH, in which R′ is a C₁ to C₁₀ alkyl radical.

Z is a single bond or a spacer group which is a linear or branched or cyclic, saturated or unsaturated C₁-C₁₀₀ and preferably C₁-C₅₀ carbon-based chain, this chain possibly being interrupted with hetero atoms such as sulfur, oxygen, nitrogen, silicon or phosphorus. It may also comprise one or more substituents such as hydroxyl, amine, thiol, carbamate, ether, acid, ester, amide, cyano or ureido groups. Preferably, it will be a polyol or a polyalkylene glycol (PEG or PPG).

Y is the function that allows the covalent bond to be established between the support molecule and the species comprising the photoactivatable functional group(s).

Y represents a function chosen from the group formed by alcohols, amines, thiols, thiosulfates, carboxylic acids and derivatives thereof such as anhydrides, acid chlorides esters, acetals and hemiacetals, aminals and hemiaminals, ketones, aldehydes, α-hydroxy ketones, α-halo ketones, epoxides, lactones, thiolactones, azalactones, isocyanate, thiocyanate, imines, imides (succinimides or glutimides), imido esters, aziridines, imidates, oxazine and oxazoline, oxazinium and oxazolinium, halogens (fluorine, chlorine, iodine or bromine), chlorotriazines, chloropyrimidines, chloroquinoxalines, chlorobenzotriazoles, sulfonyl halides (X=F, Cl, I or Br): SO₂X, siloxanes, silanols, silanes, pyridyldithio derivatives, N-hydroxysuccinimide esters, activated or nonactivated vinyls including acrylonitriles, acrylic esters and methacrylic esters, crotonic acids and esters, cinnamic acids and esters, styrenes, butadienes, vinyl ethers, vinyl ketone, maleic esters, maleimides, vinyl sulfones, hydrazines and phenyl glyoxals.

Ar represents an aromatic nucleus chosen from the group formed by:

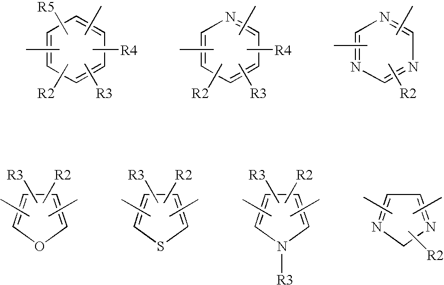

in which R₂, R₃, R₄ and R₅ represent, independently of each other, radicals chosen from the group formed by a hydrogen atom, a linear or branched C₁ to C₁₀ alkyl radical, a linear or branched C₂ to C₁₀ alkylene, a linear or branched C₂ to C₁₀ alkynyl, CF₃, CCl₃, CBr₃, NR′₃+, SR′₂+, NH₃NR′₃+, SH₂+, NH₃+, NO₂, SO₂R′, C≡N, COOH, F, Cl, Br, I, OR′, COOR′, COR′, SH, SR′, OH or SO₃H, in which R′ is a C₁ to C₁₀ alkyl radical.

The phenyl azides that may be used for the purpose of the present patent application are preferably molecules having the formula:

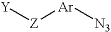

in which Y, Z and Ar have the same meanings as above.

The diazoacetates that may be used for the purpose of the present patent application are preferably molecules having the formula:

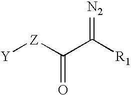

in which Y, Z and R₁ have the same meanings as above.

The diazopyruvates that may be used for the purpose of the present patent application are preferably molecules having the formula:

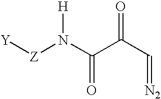

in which Y and Z have the same meanings as above.

The benzophenones that may be used for the purpose of the present patent application are preferably molecules having the formula:

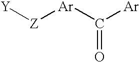

in which Y, Z and Ar have the same meanings as above.

The benzyl thio ethers that may be used for the purpose of the present patent application are preferably molecules having the formula:

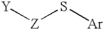

in which Y, Z and Ar have the same meanings as above.

The substituted nitrobenzenes that may be used for the purpose of the present patent application are preferably molecules having the formula:

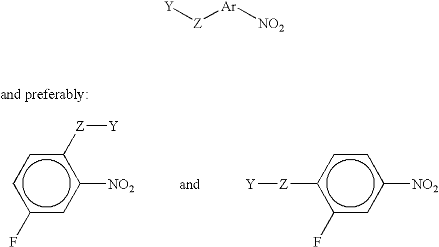

in which Y, Z and Ar have the same meanings as above.

The chemical reactions that will be carried out to synthesize the photoactivatable compound will depend on the groups Y and on the complementary groups on the support molecule, any standard chemical reaction possibly being used. Similarly, it may be envisaged to protect the groups that it is not desired to make react. Any standard reaction for protecting and deprotecting reactive functions may be used.

The photoactivatable compounds according to the invention allow the covalent grafting of the cosmetic active agents present in the medium onto the keratin material.

The grafting of the photoactivatable compound onto the cosmetic active agent, on the one hand, and onto the keratin material, on the other hand, may be performed in a single step, but it is also possible, if this procedure has advantages, to pregraft the photoactivatable compound onto the cosmetic active agent and then to graft this combination onto the keratin materials, or alternatively to graft the photoactivatable compound onto the keratin materials and then to graft this combination onto the cosmetic active agent.

Any cosmetic active agent that contains bonds capable of giving insertion reactions with the activated species derived from the photoactivatable groups may be used. These active agents may optionally contain several sites for reaction with one or more types of activated species. They may especially be natural or synthetic, water-soluble or water-insoluble polymers, mineral (metallic or nonmetallic) particles or organic particles (latices, polystyrenes or silicones), pigments, sunscreens or antioxidants.

Among the cosmetic active agents in polymer form are silicones, cationic polymers and amphoteric polymers.

Among the cosmetic active agents in the form of mineral particles are nacres, pigments or nanopigments (mean size of the primary particles: generally between 5 nm and 100 nm and preferably between 10 nm and 50 nm) of coated or uncoated metal oxides, such as, for example, nanopigments of titanium oxide (amorphous or crystallized in rutile and/or anatase form), of iron oxide, of zinc oxide, of zirconium oxide or of cerium oxide. Alumina and/or aluminum stearate are also included.

Among the cosmetic active agents in the form of sunscreens are 1,3,5-triazine derivatives, dibenzoylmethane derivatives, cinnamic derivatives, anthranilate derivatives; salicylic derivatives, camphor derivatives; benzophenone derivatives; β,β-diphenylacrylate derivatives, benzotriazole derivatives; benzalmalonate derivatives; benzimidazole derivatives; imidazolines; the bis(benzazolyl) derivatives as described in patents EP 669 323 and U.S. Pat. No. 2,463,264; p-aminobenzoic acid (PABA) derivatives; the methylenebis(hydroxyphenyl)benzotriazole derivatives as described in patent applications U.S. Pat. No. 5,237,071, U.S. Pat. No. 5,166,355, GB 2 303 549, DE 197 26 184 and EP 893 119; screening polymers and screening silicones such as those described especially in patent application WO-93/04665; dimers derived from α-alkylstyrene such as those described in patent application DE 198 55 649, 4,4-diarylbutadienes such as those described in patent applications EP 0 967 200 and DE 197 55 649.

Also among the cosmetic agents are fatty substances, softeners, antioxidants, free-radical scavengers, emollients, α-hydroxy acids, moisturizers, vitamins, insect repellants, fragrances, antiinflammatory agents, substance P antagonists, fillers and colorants.

The compositions according to the invention may also contain other constituents that do not necessarily interact with the photoactivatable compounds.

These compositions may also contain activators for the photoactivatable compounds, for instance polyamines.

The compositions according to the present invention generally comprise from 0.0001% to 50%, preferably from 0.001% to 30% and even more preferably from 0.01% to 10% by weight of photoactivatable compound, relative to the total weight of the composition.

When these compositions comprise at least one other constituent, the constituent is present in an amount of between 0.01% and 70% and preferably from 1% to 50% by weight, relative to the total weight of the composition.

The present invention also relates to a cosmetic treatment process, which consists in applying a composition containing the photoactivatable compound and, preferably, a cosmetic active agent in a suitable cosmetic solvent to the keratin materials and in exposing the keratin materials to irradiation of one or more wavelengths of between 200 and 450 nm, preferably 200 and 400 nm and even more preferably 250 and 400 nm. This process may be carried out in one or more steps.

According to another variant of said the process, it is possible to apply a composition containing the photoactivatable compound in a suitable solvent to the keratin materials, optionally to evaporate the solvent and then to apply the cosmetic active agent. The keratin materials are then exposed to irradiation of one or more wavelengths of between 200 and 450 nm, preferably 200 and 400 nm and even more preferably 250 and 400 nm, this step producing the covalent grafting of the cosmetic active agent onto the keratin materials. The crosslinking may then be performed by the further addition of an amount of photoactivatable compound to the already-grafted cosmetic active agent.

One cosmetic treatment process that is especially advantageous consists in combining the cosmetic treatment process according to the invention with a standard treatment.

This process consists in applying a cosmetic active agent to the keratin materials to be treated: these will be, for example, agents for performing a permanent-waving operation and in particular a permanent-waving reducing agent, a bleaching agent, a shampoo, a styling agent or a coloring agent (direct dye or oxidation dye), and then in applying a composition containing the photoactivatable compound according to the invention in a suitable cosmetic solvent, removing the excess solvent and irradiating the keratin materials at one or more wavelengths of between 200 and 450 nm, preferably 200 and 400 nm and even more preferably 250 and 400 nm, according to the processes in one or more steps defined above.

The examples that follow illustrate the invention without limiting its scope.

### EXAMPLE 1

Preparation of Photoactivatable Ovalbumin.

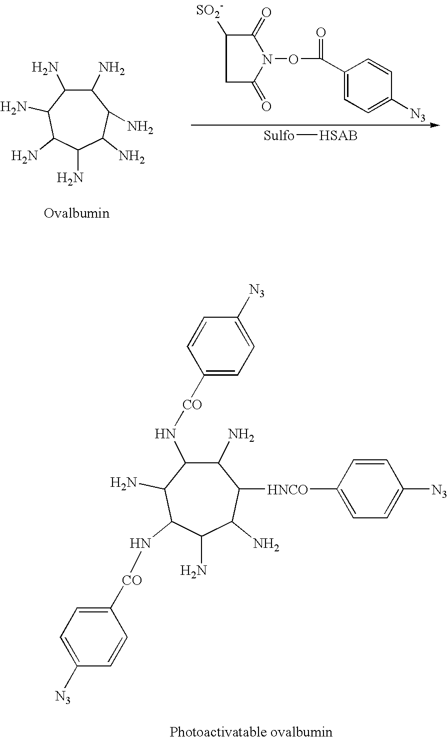

### 20 mg of Ovalbumin (Sigma) are Dissolved in 2 ml of Phosphate Buffer (pH 7.2; 0.15M) with Gentle Mechanical Stirring

2 mg of N-hydroxysulfosuccinimidyl 4-azidobenzoate (sulfo-HSAB—Pierce) are rapidly dissolved (to avoid hydrolysis) in 0.5 ml of phosphate buffer (pH 7.2; 0.15M) in another beaker.

The two solutions thus prepared are mixed together (the 2 ml of ovalbumin solution are added to the sulfo-HSAB solution). The resulting mixture is stirred for 1 hour at room temperature and protected from light. The mixture is purified on a PD-10 column (Amersham Pharmacia Biotech). 3.5 ml of purified solution are stored (after filtration using a 0.22 μm microfilter) in the form of aliquots, at −20° C. and protected from light.

### EXAMPLE 2

A. Sequential Deposition of Photoactivatable Ovalbumin and of Dextran-Fluoresceine onto Hair

A hair is taken from a lock of prewashed natural hair and attached to a glass slide with a small piece of adhesive tape at both ends. 20 μl of the ovalbumin azide solution obtained in step 1 are deposited along the entire length of the fiber.

The solution is dried in an oven (40° C.) for about 20 minutes.

20 μl of a 100 μg/ml dextran-fluoresceine solution are then deposited, the solution is dried in an oven (40° C.) for about 20 minutes and the sample is irradiated under UV (254 nm) at a power of 6 mW/cm² for 5 minutes. The hair is rinsed with a wash bottle containing demineralized water. The sample is observed by fluorescence microscopy (λ excitation=450-490 nm).

B. Deposition of Unmodified Ovalbumin and of Dextran-Fluoresceine onto Hair (Control Experiment)

A hair is taken from a lock of prewashed natural hair and attached to a glass slide with a small piece of adhesive tape arranged at both ends. 20 μl of an ovalbumin solution (10 mg/ml in phosphate buffer) are deposited along the entire length of the fiber. The solution is dried in an oven (40° C.) for about 20 minutes.

20 μl of a 100 μg/ml dextran-fluoresceine solution (control) are then deposited. The assembly is dried in an oven (40° C.) for about 20 minutes and the sample is irradiated under UV (254 nm) at a power of 6 mW/cm² for 5 minutes. The hair is rinsed with a wash bottle containing demineralized water. The sample is observed by fluorescence microscopy (λ excitation=450-490 nm).

Results

It is found that

1) The sequential deposition of unmodified ovalbumin and then of fluorescent dextran (control), followed by an irradiation, does not allow remanence of the dextran with respect to washing (no fluorescence).

2) However, after rinsing, the sample treated with the photoactivatable ovalbumin still contains fluorescent dextran at the surface. There is thus pronounced remanence.

1. Photoactivatable compound comprising a support molecule, chosen from carbon-containing and/or sulfur-containing and/or nitrogen-containing and/or phosphorus-containing nonpolymer compounds, and polymers and oligomers and to which are covalently bonded at least two photoactivatable functional species or groups containing chemical groups which, after irradiation between 200 and 450 nm, are converted into reactive species capable of reacting with chemical groups belonging to other molecules.

2. Compound according to claim 1, such that the photoactivatable functional groups are obtained by grafting compounds chosen from diazirines, phenyl azides or aryl azides, diazo compounds such as diazoacetates and diazopyruvates, benzophenones, benzyl thio ethers and substituted nitrobenzenes onto the support molecule.

3. Compound according to claim 1 or 2, such that the support molecule is a linear or branched chain containing from 1 to 80 carbon atoms and preferably 1 to 50 carbon atoms, optionally comprising 1 to 25 and preferably 1 to 10 hetero atoms (O, N, Si, S or P), this chain optionally being substituted with 1 to 60 substituents chosen from hydroxyl, amine, thiol, carbamate, ether, acid, ester, amide, cyano, ureido and halogen groups.

4. Compound according to claim 1 or 2, such that the support molecule is a polymer.

5. Compound according to claim 4, such that the support molecule is a natural polymer chosen from proteins and polysaccharides.

6. Compound according to claim 5, such that the support molecule is chosen from albumin, ovalbumin, keratin and collagen.

7. Compound according to claim 4, such that the support molecule is a synthetic polymer.

8. Compound according to claim 4 or 7, such that the support molecule is a synthetic polymer chosen from dendrimers, hyperbranched polymers and dendrons.

9. Compound according to one of claims 1 to 8, such that the support molecule has cosmetic activity.

10. Compound according to one of claims 1 to 9, such that at least one of the photoactivatable functional groups is obtained by grafting onto the support molecule a diazirine of formula: 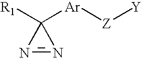
in which R₁ is chosen from the group formed by a hydrogen atom, a linear or branched C₁ to C₁₀ alkyl radical, a linear or branched C₂ to C₁₀ alkenyl radical, a linear or branched C₂ to C₁₀ alkynyl, CF₃, CCl₃, CBr₃, NR′₃+, SR′₂+, SH₂+, NH₃+, NO₂, SO₂R′, C≡N, COOH, F, Cl, Br, I, OR, COOR′, SO₃H, COR′, SH, SR′ and OH, in which R′ is a C₁ to C₁₀ alkyl radical,
Z is a single bond or a spacer group which is a linear or branched or cyclic, saturated or unsaturated C₁-C₁₀₀ and preferably C₁-C₅₀ carbon-based chain, this chain possibly being interrupted with hetero atoms such as sulfur, oxygen, nitrogen, silicon or phosphorus, and also possibly comprising one or more substituents such as hydroxyl, amine, thiol, carbamate, ether, acid, ester, amide, cyano or ureido groups,
Y represents a function chosen from the group formed by alcohols, amines, thiols, thiosulfates, carboxylic acids and derivatives thereof such as anhydrides, acid chlorides, esters, acetals and hemiacetals, aminals and hemiaminals, ketones, aldehydes, α-hydroxy ketones, α-halo ketones, epoxides, lactones, thiolactones, azalactones, isocyanate, thiocyanate, imines, imides (succinimides or glutimides), imido esters, aziridines, imidates, oxazine and oxazoline, oxazinium and oxazolinium, halogens (fluorine, chlorine, iodine or bromine), chlorotriazines, chloropyrimidines, chloroquinoxalines, chlorobenzotriazoles, sulfonyl halides (X=F, Cl, I or Br): SO₂X, siloxanes, silanols, silanes, pyridyldithio derivatives, N-hydroxysuccinimide esters, activated or nonactivated vinyls including acrylonitriles, acrylic esters and methacrylic esters, crotonic acids and esters, cinnamic acids and esters, styrenes, butadienes, vinyl ethers, vinyl ketone, maleic esters, maleimides, vinyl sulfones, hydrazines and phenyl glyoxals,
Ar represents an aromatic nucleus chosen from the group formed by: 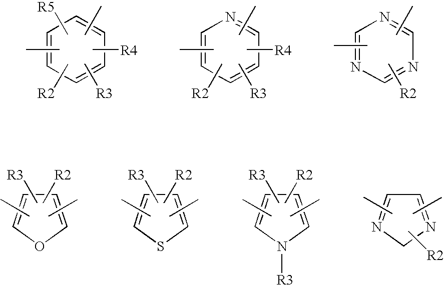
in which R₂, R₃, R₄ and R₅ represent, independently of each other, radicals chosen from the group formed by a hydrogen atom, a linear or branched C₁ to C₁₀ alkyl radical, a linear or branched C₂ to C₁₀ alkenyl radical, a linear or branched C₂ to C₁₀ alkynyl, CF₃, CCl₃, CBr₃, NR′₃+, SR′₂+, SH₂+, NH₃+, NO₂, SO₂R′, C≡N, COOH, F, Cl, Br, I, OR′, COOR′, COR′, SH, SR′, OH or SO₃H, in which R′ is a C₁ to C₁₀ alkyl radical.

11. Compound according to one of claims 1 to 9, such that at least one of the photoactivatable functional groups is obtained by grafting onto the support molecule a phenyl azide of formula: 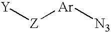
such that:
Z is a single bond or a spacer group which is a linear or branched or cyclic, saturated or unsaturated C₁-C₁₀₀ and preferably C₁-C₅₀ carbon-based chain, this chain possibly being interrupted with hetero atoms such as sulfur, oxygen, nitrogen, silicon or phosphorus, and also possibly comprisising one or more substituents such as hydroxyl, amine, thiol, carbamate, ether, acid, ester, amide, cyano or ureido groups,
Y represents a function chosen from the group formed by alcohols, amines, thiols, thiosulfates, carboxylic acids and derivatives thereof such as anhydrides, acid chlorides, esters, acetals and hemiacetals, aminals and hemiaminals, ketones, aldehydes, α-hydroxy ketones, α-halo ketones, epoxides, lactones, thiolactones, azalactones, isocyanate, thiocyanate, imines, imides (succinimides or glutimides), imido esters, aziridines, imidates, oxazine and oxazoline, oxazinium and oxazolinium, halogens (fluorine, chlorine, iodine or bromine), chlorotriazines, chloropyrimidines, chloroquinoxalines, chlorobenzotriazoles, sulfonyl halides (X=F, Cl, I or Br): SO₂X, siloxanes, silanols, silanes, pyridyldithio derivatives, N-hydroxysuccinimide esters, activated or nonactivated vinyls including acrylonitriles, acrylic esters and methacrylic esters, crotonic acids and esters, cinnamic acids and esters, styrenes, butadienes, vinyl ethers, vinyl ketone, maleic esters, maleimides, vinyl sulfones, hydrazines and phenyl glyoxals,
Ar represents an aromatic nucleus chosen from the group formed by: 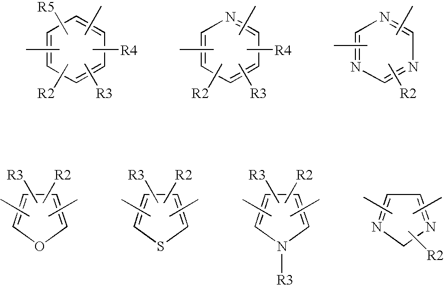
in which R₂, R₃, R₄ and R₅ represent, independently of each other, radicals chosen from the group formed by a hydrogen atom, a linear or branched C₁ to C₁₀ alkyl radical, a linear or branched C₂ to C₁₀ alkenyl radical, a linear or branched C₂ to C₁₀ alkynyl, CF₃, CCl₃, CBr₃, NR′₃+, SR′₂+, SH₂+, NH₃+, NO₂, SO₂R′, C≡N, COOH, F, Cl, Br, I, OR′, COOR′, COR′, SH, SR′, OH or SO₃H, in which R′ is a C₁ to C₁₀ alkyl radical.

12. Compound according to one of claims 1 to 9, such that at least one of the photoactivatable functional groups is obtained by grafting onto the support molecule a diazoacetate of formula: 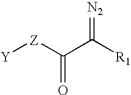
in which R₁ is chosen from the group formed by a hydrogen atom, a linear or branched C₁ to C₁₀ alkyl radical, a linear or branched C₂ to C₁₀ alkenyl radical, a linear or branched C₂ to C₁₀ alkynyl, CF₃, CCl₃, CBr₃, NR′₃+, SR′₂+, SH₂+, NH₃+, NO₂, SO₂R′, C≡N, COOH, F, Cl, Br, I, OR, COOR′, SO₃H, COR′, SH, SR′ and OH, in which R′ is a C₁ to C₁₀ alkyl radical,
Z is a single bond or a spacer group which is a linear or branched or cyclic, saturated or unsaturated C₁-C₁₀₀ and preferably C₁-C₅₀ carbon-based chain, this chain possibly being interrupted with hetero atoms such as sulfur, oxygen, nitrogen, silicon or phosphorus, and also possibly comprising one or more substituents such as hydroxyl, amine, thiol, carbamate, ether, acid, ester, amide, cyano or ureido groups,
Y represents a function chosen from the group formed by alcohols, amines, thiols, thiosulfates, carboxylic acids and derivatives thereof such as anhydrides, acid chlorides, esters, acetals and hemiacetals, aminals and hemiaminals, ketones, aldehydes, α-hydroxy ketones, α-halo ketones, epoxides, lactones, thiolactones, azalactones, isocyanate, thiocyanate, imines, imides (succinimides or glutimides), imido esters, aziridines, imidates, oxazine and oxazoline, oxazinium and oxazolinium, halogens (fluorine, chlorine, iodine or bromine), chlorotriazines, chloropyrimidines, chloroquinoxalines, chlorobenzotriazoles, sulfonyl halides (X=F, Cl, I or Br): SO₂X, siloxanes, silanols, silanes, pyridyldithio derivatives, N-hydroxysuccinimide esters, activated or nonactivated vinyls including acrylonitriles, acrylic esters and methacrylic esters, crotonic acids and esters, cinnamic acids and esters, styrenes, butadienes, vinyl ethers, vinyl ketone, maleic esters, maleimides, vinyl sulfones, hydrazines and phenyl glyoxals.

13. Compound according to one of claims 1 to 9, such that at least one of the photoactivatable functional groups is obtained by grafting onto the support molecule a diazopyruvate of formula: 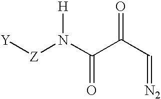
in which
Z is a single bond or a spacer group which is a linear or branched or cyclic, saturated or unsaturated C₁-C₁₀₀ and preferably C₁-C₅₀ carbon-based chain, this chain possibly being interrupted with hetero atoms such as sulfur, oxygen, nitrogen, silicon or phosphorus, and also possibly comprising one or more substituents such as hydroxyl, amine, thiol, carbamate, ether, acid, ester, amide, cyano or ureido groups,
Y represents a function chosen from the group formed by alcohols, amines, thiols, thiosulfates, carboxylic acids and derivatives thereof such as anhydrides, acid chlorides, esters, acetals and hemiacetals, aminals and hemiaminals, ketones, aldehydes, α-hydroxy ketones, α-halo ketones, epoxides, lactones, thiolactones, azalactones, isocyanate, thiocyanate, imines, imides (succinimides or glutimides), imido esters, aziridines, imidates, oxazine and oxazoline, oxazinium and oxazolinium, halogens (fluorine, chlorine, iodine or bromine), chlorotriazines, chloropyrimidines, chloroquinoxalines, chlorobenzotriazoles, sulfonyl halides (X=F, Cl, I or Br): SO₂X, siloxanes, silanols, silanes, pyridyldithio derivatives, N-hydroxysuccinimide esters, activated or nonactivated vinyls including acrylonitriles, acrylic esters and methacrylic esters, crotonic acids and esters, cinnamic acids and esters, styrenes, butadienes, vinyl ethers, vinyl ketone, maleic esters, maleimides, vinyl sulfones, hydrazines and phenyl glyoxals.

14. Compound according to one of claims 1 to 9, such that at least one of the photoactivatable functional groups is obtained by grafting onto the support molecule a benzophenone of formula: 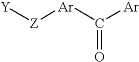
such that:
Z is a single bond or a spacer group which is a linear or branched or cyclic, saturated or unsaturated C₁-C₁₀₀ and preferably C₁-C₅₀ carbon-based chain, this chain possibly being interrupted with hetero atoms such as sulfur, oxygen, nitrogen, silicon or phosphorus, and also possibly comprising one or more substituents such as hydroxyl, amine, thiol, carbamate, ether, acid, ester, amide, cyano or ureido groups,
Y represents a function chosen from the group formed by alcohols, amines, thiols, thiosulfates, carboxylic acids and derivatives thereof such as anhydrides, acid chlorides, esters, acetals and hemiacetals, aminals and hemiaminals, ketones, aldehydes, α-hydroxy ketones, α-halo ketones, epoxides, lactones, thiolactones, azalactones, isocyanate, thiocyanate, imines, imides (succinimides or glutimides), imido esters, aziridines, imidates, oxazine and oxazoline, oxazinium and oxazolinium, halogens (fluorine, chlorine, iodine or bromine), chlorotriazines, chloropyrimidines, chloroquinoxalines, chlorobenzotriazoles, sulfonyl halides (X=F, Cl, I or Br): SO₂X, siloxanes, silanols, silanes, pyridyldithio derivatives, N-hydroxysuccinimide esters, activated or nonactivated vinyls including acrylonitriles, acrylic esters and methacrylic esters, crotonic acids and esters, cinnamic acids and esters, styrenes, butadienes, vinyl ethers, vinyl ketone, maleic esters, maleimides, vinyl sulfones, hydrazines and phenyl glyoxals,
Ar represents an aromatic nucleus chosen from the group formed by: 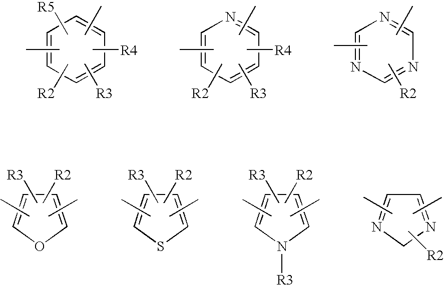
in which R₂, R₃, R₄ and R₅ represent, independently of each other, radicals chosen from the group formed by a hydrogen atom, a linear or branched C₁ to C₁₀ alkyl radical, a linear or branched C₂ to C₁₀ alkenyl radical, a linear or branched C₂ to C₁₀ alkynyl, CF₃, CCl₃, CBr₃, NR′₃+, SR′₂+, SH₂+, NH₃+, NO₂, SO₂R′, C≡N, COOH, F, Cl, Br, I, OR′, COOR′, COR′, SH, SR′, OH or SO₃H, in which R′ is a C₁ to C₁₀ alkyl radical.

15. Compound according to one of claims 1 to 9, such that at least one of the photoactivatable functional groups is obtained by grafting onto the support molecule a benzyl thio ether of formula: 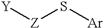
such that:
Z is a single bond or a spacer group which is a linear or branched or cyclic, saturated or unsaturated C₁-C₁₀₀ and preferably C₁-C₅₀ carbon-based chain, this chain possibly being interrupted with hetero atoms such as sulfur, oxygen, nitrogen, silicon or phosphorus, and also possibly comprising one or more substituents such as hydroxyl, amine, thiol, carbamate, ether, acid, ester, amide, cyano or ureido groups,
Y represents a function chosen from the group formed by alcohols, amines, thiols, thiosulfates, carboxylic acids and derivatives thereof such as anhydrides, acid chlorides, esters, acetals and hemiacetals, aminals and hemiaminals, ketones, aldehydes, α-hydroxy ketones, α-halo ketones, epoxides, lactones, thiolactones, azalactones, isocyanate, thiocyanate, imines, imides (succinimides or glutimides), imido esters, aziridines, imidates, oxazine and oxazoline, oxazinium and oxazolinium, halogens (fluorine, chlorine, iodine or bromine), chlorotriazines, chloropyrimidines, chloroquinoxalines, chlorobenzotriazoles, sulfonyl halides (X=F, Cl, I or Br): SO₂X, siloxanes, silanols, silanes, pyridyldithio derivatives, N-hydroxysuccinimide esters, activated or nonactivated vinyls including acrylonitriles, acrylic esters and methacrylic esters, crotonic acids and esters, cinnamic acids and esters, styrenes, butadienes, vinyl ethers, vinyl ketone, maleic esters, maleimides, vinyl sulfones, hydrazines and phenyl glyoxals,
Ar represents an aromatic nucleus chosen from the group formed by: 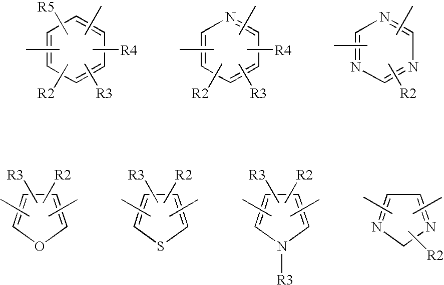
in which R₂, R₃, R₄ and R₅ represent, independently of each other, radicals chosen from the group formed by a hydrogen atom, a linear or branched C₁ to C₁₀ alkyl radical, a linear or branched C₂ to C₁₀ alkenyl radical, a linear or branched C₂ to C₁₀ alkynyl, CF₃, CCl₃, CBr₃, NR′₃+, SR′₂+, SH₂+, NH₃+, NO₂, SO₂R′, C≡N, COOH, F, Cl, Br, I, OR′, COOR′, COR′, SH, SR′, OH or SO₃H, in which R′ is a C₁ to C₁₀ alkyl radical.

16. Compound according to one of claims 1 to 9, such that at least one of the photoactivatable functional groups is obtained by grafting onto the support molecule a substituted nitrobenzene of formula: 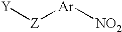
such that:
Z is a single bond or a spacer group which is a linear or branched or cyclic, saturated or unsaturated C₁-C₁₀₀ and preferably C₁-C₅₀ carbon-based chain, this chain possibly being interrupted with hetero atoms such as sulfur, oxygen, nitrogen, silicon or phosphorus, and also possibly comprising one or more substituents such as hydroxyl, amine, thiol, carbamate, ether, acid, ester, amide, cyano or ureido groups,
Y represents a function chosen from the group formed by alcohols, amines, thiols, thiosulfates, carboxylic acids and derivatives thereof such as anhydrides, acid chlorides, esters, acetals and hemiacetals, aminals and hemiaminals, ketones, aldehydes, α-hydroxy ketones, α-halo ketones, epoxides, lactones, thiolactones, azalactones, isocyanate, thiocyanate, imines, imides (succinimides or glutimides), imido esters, aziridines, imidates, oxazine and oxazoline, oxazinium and oxazolinium, halogens (fluorine, chlorine, iodine or bromine), chlorotriazines, chloropyrimidines, chloroquinoxalines, chlorobenzotriazoles, sulfonyl halides (X=F, Cl, I or Br): SO₂X, siloxanes, silanols, silanes, pyridyldithio derivatives, N-hydroxysuccinimide esters, activated or nonactivated vinyls including acrylonitriles, acrylic esters and methacrylic esters, crotonic acids and esters, cinnamic acids and esters, styrenes, butadienes, vinyl ethers, vinyl ketone, maleic esters, maleimides, vinyl sulfones, hydrazines and phenyl glyoxals,
Ar represents an aromatic nucleus chosen from the group formed by: 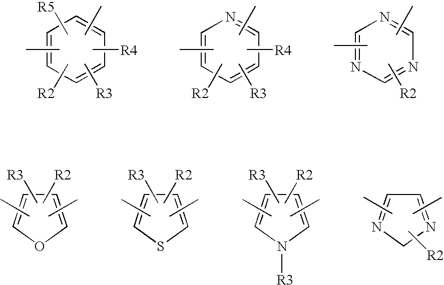
in which R₂, R₃, R₄ and R₅ represent, independently of each other, radicals chosen from the group formed by a hydrogen atom, a linear or branched C₁ to C₁₀ alkyl radical, a linear or branched C₂ to C₁₀ alkenyl radical, a linear or branched C₂ to C₁₀ alkynyl, CF₃, CCl₃, CBr₃, NR′₃+, SR′₂+, SH₂+, NH₃+, NO₂, SO₂R′, C≡N, COOH, F, Cl, Br, I, OR′, COOR′, COR′, SH, SR′, OH or SO₃H, in which R′ is a C₁ to C₁₀ alkyl radical.

17. Compound according to claim 16, such that the substituted nitrobenzene compound is chosen from:

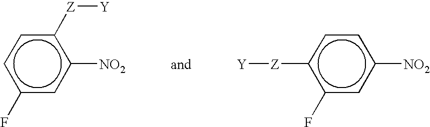

18. Cosmetic composition containing, in a cosmetically acceptable solvent, at least one photoactivatable compound according to one of claims 1 to 17.

19. Cosmetic composition according to claim 18, containing at least one cosmetic active agent.

20. Cosmetic composition according to claim 19, containing a cosmetic active agent chosen from the group formed by polymers, mineral or organic particles, sunscreens, antioxidants, colorants, fatty substances, softeners, antioxidants, free-radical scavengers, emollients, α-hydroxy acids, moisturizers, vitamins, insect repellants, fragrances, antiinflammatory agents, substance P antagonists and fillers.

21. Cosmetic composition according to claim 19 or 20, such that the photoactivatable compound and the cosmetic active agent are covalently bonded.

22. Cosmetic composition according to claim 19 or 20, such that the photoactivatable compound and the cosmetic active agent are present in the form of a mixture.

23. Use of the cosmetic composition according to one of claims 18 to 22, as a coloring agent, as a moisturizer, as an agent for increasing the sheen, especially of the hair (sheen agent), as a sunscreen, as a conditioner or as an agent for shaping keratin fibers.

24. Cosmetic treatment process, characterized in that it consists in applying to keratin materials a cosmetic composition according to one of claims 17 to 20 and in exposing said keratin materials to a radiation of one or more wavelengths of between 200 et 450 nm, preferably 200 and 400 nm and even more preferably 250 and 400 nm.

25. Process according to claim 24, such that it is a one-step process.

26. Process according to claim 24, such that it consists in applying a composition containing the photoactivatable compound in a suitable solvent to keratin materials, evaporating the solvent, applying the cosmetic active agent and then irradiating the keratin materials.

27. Process according to claim 26, such that it consists in applying a further addition of an amount of photoactivatable compound to the already-grafted cosmetic active agent.

28. Process according to claim 24, such that it consists in applying a cosmetic agent and then a composition containing the photoactivatable compound in a suitable solvent, removing the excess solvent and irradiating the keratin materials.
